SEQUENCE LISTING (20 sequences)

SEQ ID NO: 1 - DNA, 20 bp, Artificial Sequence
  Fully synthetic
gggactcactcacctgtacg

SEQ ID NO: 2 - DNA, 21 bp, Artificial Sequence
  Fully synthetic
ggggactcactcacctgtaca

SEQ ID NO: 3 - DNA, 20 bp, Artificial Sequence
  Fully synthetic
tgggttaaaacaggcacaga

SEQ ID NO: 4 - DNA, 20 bp, Artificial Sequence
  Fully synthetic
ctattgggaaagtcactgcg

SEQ ID NO: 5 - DNA, 21 bp, Artificial Sequence
  Fully synthetic
cctattgggaaagtcactgca

SEQ ID NO: 6 - DNA, 20 bp, Artificial Sequence
  Fully synthetic
cccatcacattgaacagacc

SEQ ID NO: 7 - DNA, 21 bp, Artificial Sequence
  Fully synthetic
atcagcaaatgcaaagaaggt

SEQ ID NO: 8 - DNA, 19 bp, Artificial Sequence
  Fully synthetic
cagcaaatgcaaagaaggg

SEQ ID NO: 9 - DNA, 22 bp, Artificial Sequence
  Fully synthetic
cactaaaattttctgccaccat

SEQ ID NO: 10 - DNA, 27 bp, Artificial Sequence
  Fully synthetic
cagatcaataaggtgttttcttacagg

SEQ ID NO: 11 - DNA, 27 bp, Artificial Sequence
  Fully synthetic
cagatcaataaggtgttttcttacaga

SEQ ID NO: 12 - DNA, 21 bp, Artificial Sequence
  Fully synthetic
tgtcttcagggagacctgtgt

SEQ ID NO: 13 - DNA, 22 bp, Artificial Sequence
  Fully synthetic
ttaagccaaagaataggcacaa

SEQ ID NO: 14 - DNA, 22 bp, Artificial Sequence
  Fully synthetic
ttaagccaaagaataggcacag

SEQ ID NO: 15 - DNA, 21 bp, Artificial Sequence
  Fully synthetic
ccccgagaatttattgaaagg

SEQ ID NO: 16 - DNA, 23 bp, Artificial Sequence
  Fully synthetic
aataaacctccttggcctgatag

SEQ ID NO: 17 - DNA, 19 bp, Artificial Sequence
  Fully synthetic
cctccttggcctgattgat

SEQ ID NO: 18 - DNA, 26 bp, Artificial Sequence
  Fully synthetic
tggcctaatatgtaacttctctttga

SEQ ID NO: 19 - DNA, 30 bp, Artificial Sequence
  Fully synthetic
ccagcctctctcatgcttttggccagacag

SEQ ID NO: 20 - DNA, 30 bp, Artificial Sequence
  Fully synthetic
gggtcgaggacagttccgtgtagaagtgga

(19) United States
(12) Patent Application Publication
(10) Pub. No.: US 2022/0170103 A1
(43) Pub. Date: Jun. 2, 2022

(54) GENOTYPING KIT FOR DETECTION OF SIX CD36 MUTANT GENES THAT ENCODE GPIV DEFICIENCY

(71) Applicant: Nanning Blood Center (Nanning Institute of Transfusion Medicine), Nanning (CN)

(72) Inventors: Lilan LI, Nanning (CN); Guoguang WU, Nanning (CN)

(21) Appl. No.: 17/673,769

(22) Filed: Feb. 16, 2022

Related U.S. Application Data
(63) Continuation-in-part of application No. PCT/CN2020/116567, filed on Sep. 21, 2020.

(30) Foreign Application Priority Data
Sep. 25, 2019 (CN) 201910908778.5

(51) Int. Cl. C12Q 1/6883

(52) U.S. Cl. CPC C12Q 1/6883; C12Q 2600/166; C12Q 2600/156

(57) ABSTRACT

The disclosure provides a genotyping kit for detection of six CD36 mutant genes that encode GPIV deficiency. The kit includes six primers configured to amplify a sequence of CD36 wile-type gene, six primers configured to amplify a sequence of CD36 mutant genes, and six universal primers. The mutations of CD36 gene include the following mutation sites: C275T (Thr92Met), 730G>A (Asp244Asn), Exon-10+2 T>G (Change in splicing site), 1123C>T (Pro375Ser), 1229T>C (Ile410Thr), and 1332 ints TGAT (frameshift at AA 445).

### CROSS-REFERENCE TO RELATED APPLICATIONS

This application is a continuation-in-part of International Patent Application No. PCT/CN2020/116567 with an international filing date of Sep. 21, 2020, designating the United States, now pending, and further claims foreign priority benefits to Chinese Patent Application No. 201910908778.5 filed Sep. 25, 2019. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of biomedical detection, and more particularly, to a genotyping kit for detection of six CD36 mutant genes that encode GPIV deficiency.

Platelet glycoprotein IV, also known as GPIV, CD36, GP88, GPIIIb FAT or SCARB3, is a transmembrane receptor that belongs to the class B scavenger receptor family. GPIV can be glycosylated extensively and has a molecular mass of almost 88,000 D. Human GPIV has 472 amino acids. GPIV performs a number of critical functions as thrombin receptor, collagen receptor, long-chain-fatty-acid receptor, etc. GPIV has shown to be involved in inducing apoptosis, removing oxidized low-density lipoprotein in blood plasma, promoting the adhesion of abnormal shaped red blood cells, etc. Numerous studies have demonstrated that GPIV is involved in the development of hemostasis, thrombosis, alloimmune platelet disorders, hypercholesterolemia, obesity, peripheral atherosclerosis, arterial hypertension, cardiomyopathy, diabetes, malaria, presenile dementia (Alzheimer's disease), cancer and other diseases.

Some individuals may have GPIV deficiency in platelets or/and monocytes. GPIV deficiency can be divided into two phenotypes: Type I deficiency, that is, GPIV is absent in both of platelets and monocytes; Type II deficiency, that is, GPIV is absent in platelets, but present in monocytes. Mutations of CD36 gene expressing GPIV is an important reason for the deficiency of human GPIV antigen. CD36 gene is located at q11.2 on chromosome 7 and contains 15 exons spanning over 32,000 bp. Exons 3 to 14 form a coding region of the GPIV. GPIV deficiency in different populations may be caused by different mutations due to population polymorphism. In the population of Guangxi, China, 6 novel mutant genes occur leading to the GPIV deficiency, comprising C275T (Thr92Met), 730G>A (Asp244Asn), Exon-10+2 T>G (Change in splicing site), 1123C>T (Pro375Ser), 1229T>C (Ile410Thr) and 1332ints TGAT (frameshift at AA 445), the detailed information of them were show in Table 1. Detecting and screening of polymorphisms of the 6 mutant genes may help to comprehensively master the distribution characteristics of individuals with GPIV deficiency caused by these 6 GPIV mutant genes in the population and their impact on individual physiological and pathological functions, further mastering the polymorphism and genetic characteristics of the six mutant genes in the population.

TABLE 1
Detailed information of six CD36 mutant genes that encode GPIV deficiency

| | CD36 | Mutant site of | Induced CD36 | Amino acid |
|---|---|---|---|---|
| No. | mutation | CD36 DNA ^{a} | mRNA changes ^{b} | change ^{c} |
| 1 | C275T | c.275C > T | c.275C > T | p. (Thr92Met) |
| | (Thr92Met) | | | |
| 2 | 730G > A | c.730G > A | c.730G > A | p. (Asp244Asn) |
| | (Asp244Asn) | | | |
| 3 | Exon-10 + 2 T > | c.1006 + 2T > | c.819_1006de1. | p.(Ser274GlufsTer16) |
| | G (Change in | G | | |
| | splicing site) | | | |
| 4 | 1123C > T | c.1123C > T | c.1123C > 2T | p. (Pro375Ser) |
| | (Pro375Ser) | | | |
| 5 | 1229T > C | c.1229T > C | c.1229T > C | p. (Ile410Thr) |
| | (Ile410Thr) | | | |
| 6 | 1332 ints TGAT | c.1331_1332 | c.1331_1332insTGAT | p.(G1u445Aspfs |
| | (frameshift at | | | Ter64) |
| | AA 445) | insTGAT | | |

^{a} NCBI reference sequence: NG_008192.1
^{b} NCBI reference sequence: NM_000072.3
^{c} NCBI reference sequence: NP_000063.2

To screen the GPIV-deficiency individuals in the populations, the GPIV-deficiency individuals are often typed by serotyping or gene sequencing, sometimes by sequence specific primer-PCR (SSP-PCR), PCR-restriction fragment length polymorphism (PCR-RFLP), TaqMan probe-based RQ-PCR analysis. The mutant gene is detected and compared with the wild type to identify individuals with GPIV-deficiency. The detection technology used is often cumbersome and non-specific and cannot be used for specific, high-throughput screening and identification of individuals with GPIV-deficiency caused by the specific mutant genes. It is also impossible to conduct targeted, group testing, analysis and research on individuals with GPIV deficiency caused by the six CD36 mutant genes.

### SUMMARY

The disclosure provides a genotyping kit for detection of six CD36 mutant genes that encode GPIV deficiency. The kit uses PCR-SSP technique to identify the genotypes of the GPIV deficiency caused by mutations of CD36 gene, comprising C275T (Thr92Met), 730G>A (Asp244Asn), Exon-10+2 T>G (Change in splicing site), 1123C>T (Pro375Ser), 1229T>C (Ile410Thr), and 1332 ints TGAT (frameshift at AA 445).

Specifically, the kit comprises: wild-type sequence specific primer and mutant-type sequence specific primers for each mutant site of the six CD36 mutations that encode GPIV deficiency, and their universal primer; the mutations of CD36 gene comprises following mutation sites: C275T (Thr92Met), 730G>A (Asp244Asn), Exon-10+2 T>G (Change in splicing site), 1123C>T (Pro375Ser), 1229T>C (Ile410Thr), and 1332 ints TGAT (frameshift at AA 445).

A genotyping method for detection of six CD36 mutations that encode GPIV deficiency using the kit comprises:

1) amplifying a nucleotide sequence of human C-reactive protein (CRP) with a pair of polymerase chain reaction (PCR) primers; using the amplified product as an internal reference for each PCR system;

2) each PCR system for detection of CD36 mutations that encode GPIV deficiency comprising: wild-type sequence specific primer and mutant-type sequence specific primer for each mutant site of the CD36 mutation that encodes GPIV deficiency, and a universal primer; identifying the CD36 genotype of the individuals with GPIV deficiency with a corresponding PCR system, and the mutations of CD36 gene comprising following mutation sites: C275T (Thr92Met), 730G>A (Asp244Asn), Exon-10+2 T>G (Change in splicing site), 1123C>T (Pro375Ser), 1229T>C (Ile410Thr), and 1332 ints TGAT (frameshift at AA 445);

3) amplifying the wild-type sequence and the mutated sequence of each mutation site through two corresponding PCR systems, respectively; genotyping each individual with GPIV deficiency caused by the six mutations in the CD36 gene, where all of the PCR systems may be amplified under the same amplification conditions; separating PCR products through electrophoresis on a 2% agarose gel; and to observe and analyze the detection results.

Table 2 shows detailed information of PCR primers used in genotyping of six CD36 mutant genes that encode GPIV deficiency.

TABLE 2
Information of internal reference primer and sequence-
specific primers (PCR-SSP) used in genotyping of six
CD36 mutant genes that encode GPIV deficiency

| | | | | | PCR | |
|---|---|---|---|---|---|---|
| | | Primer | Forward/ | Primer | product | SEQ |
| Mutant | | character- | Reverse | sequences | size | ID |
| genotypes | Primers | istics | Primers | (5′-3′) | (bp) | NO |
| C275T | GPIV-275a | 275C wild- | Reverse | 5′- GGG ACT | 135 | 1 |
| | | type primer | | CAC TCA CCT | | |
| | | | | GTA CG -3′ | | |
| | GPIV-275b | 275T mutant | Reverse | 5′- GGG GAC | 136 | 2 |
| | | primer | | TCA CTC ACC | | |
| | | | | TGT ACA -3′ | | |
| | GPIV-275c | Universal | Forward | 5′- TGG GTT | | 3 |
| | | primer | | AAA ACA GGC | | |
| | | | | ACA GA -3′ | | |
| G730A | GPIV-730a | 730G wild- | Forward | 5′- CTA TTG | 216 | 4 |
| | | type primer | | GGA AAG TCA | | |
| | | | | CTG CG -3′ | | |
| | GPIV-730b | 730A mutant | Forward | 5′- CCT ATT | 217 | 5 |
| | | primer | | GGG AAA GTC | | |
| | | | | ACT GCA-3′ | | |
| | GPIV-730c | Universal | Reverse | 5′- CCC ATC | | 6 |
| | | primer | | ACA TTG AAC | | |
| | | | | AGA CC-3′ | | |
| Exon-10 | GPIV- | E10 + 2T | Forward | 5′- ATC AGC | 243 | 7 |
| (+2T>G) | E10(+2)a | wild-type | | AAA TGC AAA | | |
| | | primer | | GAA GGT -3′ | | |
| | GPIV- | E10 + 2G | Forward | 5′- CAG CAA | 241 | 8 |
| | E10(+2)b | mutant | | ATG CAA AGA | | |
| | | primer | | AGG G -3′ | | |
| | GPIV- | Universal | Reverse | 5′- CAC TAA | | 9 |
| | E10(+2)c | primer | | AAT TTT CTG | | |
| | | | | CCA CCA | | |
| | | | | T-3′ | | |
| C1123T | GPIV-1123a | 1123C wild- | Reverse | 5′- CAG ATC | 152 | 10 |
| | | type primer | | AAT AAG GTG | | |
| | | | | TTT TCT TAC | | |
| | | | | AGG-3′ | | |
| | GPIV-1123b | 1123T | Reverse | 5′- CAG ATC | 152 | 11 |
| | | mutant | | AAT AAG GTG | | |
| | | primer | | TTT TCT TAC | | |
| | | | | AGA -3′ | | |
| | GPIV-1123c | Universal | Forward | 5′- TGT CTT | | 12 |
| | | primer | | CAG GGA GAC | | |
| | | | | CTG TGT-3′ | | |
| T1229C | GPIV-1229a | 1229T wild- | Reverse | 5′- TTA AGC | 283 | 13 |
| | | type primer | | CAA AGA ATA | | |
| | | | | GGC ACA | | |
| | | | | A-3′ | | |
| | GPIV-1229b | 1229C | Reverse | 5′- TTA AGC | 283 | 14 |
| | | mutant | | CAA AGA ATA | | |
| | | primer | | GGC ACA | | |
| | | | | G-3′ | | |
| | GPIV-1229c | Universal | Forward | 5′- CCC CGA | | 15 |
| | | primer | | GAA TTT ATT | | |
| | | | | GAA AGG -3′ | | |
| 1332 ints | GPIV-1332a | 1332 wild- | Forward | 5′- AAT AAA | 200 | 16 |
| TGAT | | type primer | | CCT CCT TGG | | |
| | | | | CCT GAT | | |
| | | | | AG -3′ | | |
| | GPIV-1332b | 1332 ints | Forward | 5′- CCT CCT | 198 | 17 |
| | | TGAT wild- | | TGG CCT GAT | | |
| | | type primer | | TGA T-3′ | | |
| | GPIV-1332c | Universal | Reverse | 5′- TGG CCT | | 18 |
| | | primer | | AAT ATG TAA | | |
| | | | | CTT CTC TTT | | |
| | | | | GA -3′ | | |
| Internal | CRP I | Internal | Forward | 5′- CCA GCC | 440 | 19 |
| Reference | | reference | | TCT CTC ATG | | |
| CRP | | primers | | CTT TTG GCC | | |
| | | | | AGA CAG-3′ | | |
| | CRP II | | Reverse | 5′- GGG TCG | | 20 |
| | | | | AGG ACA GTT | | |
| | | | | CCG TGT AGA | | |
| | | | | AGT GGA-3′ | | |

A method for amplifying the sequence with regard to the mutation of CD36 gene comprises: amplifying the wild-type sequence by PCR with the wild-type primer a and the universal primer c, amplifying the mutant sequence by PCR with the mutant primer b and the universal primer c; and amplifying the internal reference using the forward primer CRPI and the reverse primer CRP II. Three PCR systems are used for amplification of different mutated sequences. Table 3 shows the compositions of the three PCR systems.

TABLE 3
Compositions of PCR systems (Final volume: 10 μL per tube)

| PCR system | A | | B | | C | |
|---|---|---|---|---|---|---|
| Number of tubes | 1 | tube | 1 | tube | 1 | tube |
| H₂O | 4.895 | μL | 4.295 | μL | 5.095 | μL |
| 10 × Buffer (Mg²⁺ free) | 1 | μL | 1 | μL | 1 | μL |
| MgCl₂ (25 mM) | 0.8 | μL | 1.4 | μL | 0.6 | μL |
| dNTPs (2.5 mM each) | 0.8 | μL | 0.8 | μL | 0.8 | μL |
| rTaq DNA Polmerase (Takara) | 0.075 | μL | 0.075 | μL | 0.075 | μL |
| CRPI + CRPII (5 μM each) | 0.15 | μL | 0.15 | μL | 0.15 | μL |
| Sequence-specific primer | 0.5 | μL | 0.5 | μL | 0.5 | μL |
| a or b (10 μM) | | | | | | |
| Universal primer c (10 μM) | 0.5 | μL | 0.5 | μL | 0.5 | μL |
| DNA | 1 | μL | 1 | μL | 1 | μL |
| Cresyl red (10 mg/m1) | 0.04 | μL | 0.04 | μL | 0.04 | μL |
| Glycerine 50% (v/v) | 0.24 | μL | 0.24 | μL | 0.24 | μL |
| Final concentration of MgCl₂ | 2 | mM | 3.5 | mM | 1.5 | mM |

An appropriate range of magnesium ion (Mg²⁺) concentration is one of the basic conditions to have a desired effect on PCR. Mg²⁺ concentration is necessary for Taq DNA polymerase and affects primer annealing, melting temperature of the template and the PCR product, specificity of PCR, primer dimer formation. The enzyme activity reduces at low Mg²⁺ concentration and high Mg²⁺ concentration would lead to non-specific amplification. Therefore, the disclosure examines the influence of different Mg²⁺ concentrations on the PCR system for detection of different mutant genotypes. The results indicate that the PCR effect is best when C275T and Exon-10 (+2 T>G) PCR systems contain Mg²⁺ at a final concentration of 2 mM, T1229C and 1332 ints TGAT PCR systems contain Mg²⁺ at a final concentration of 3.5 mM, and G730A and C1123T amplification systems contain Mg²⁺ at a final concentration of 1.5 mM. Table 4 shows the optimum final Mg²⁺ concentration for amplification of the mutant gene that comprises the target SNP site.

TABLE 4
Optimum final Mg²⁺concentration for amplification
of the mutant gene that comprises the target SNP site

| PCR | Final Mg²⁺ | | |
|---|---|---|---|
| system | concentration | | Applicable mutation type |
| system A | 2 | mM | C275T, Exon-10 (+2 T > G) |
| system B | 3.5 | mM | T1229C, 1332 ints TGAT |
| system C | 1.5 | mM | G730A, C1123T |

PCR amplification is carried out by a thermocycler. All of the systems can be carried out under the same conditions: 95° C. for 5 min, 25 cycles of 95° C. for 30 sec, 68° C.-0.4° C./cycle for 30 sec, and 72° C. for 30 sec, 15 cycles of 95° C. for 30 sec, 54° C. for 30 sec, and 72° C. for 30 sec, with the final elongation step at 72° C. for 5 min, followed by an infinite hold at 12° C.

PCR products are separated for electrophoresis on a 2% agarose gel.

The following advantages are associated with the human CD36 genotyping kit of the disclosure: the disclosure uses the molecular basis of human GPIV deficiency and the principle of PCR-SSP to provide a genotyping method for CD36 mutant genes that encode GPIV deficiency. The genotyping method can identify six mutations of CD36 gene that lead to GPIV deficiency, comprising C275T (Thr92Met), 730G>A (Asp244Asn), Exon-10+2 T>G (Change in splicing site), 1123C>T (Pro375Ser), 1229T>C (Ile410Thr), and 1332 ints TGAT (frameshift at AA 445). The kit of the disclosure is used for genotyping the individuals with GPIV deficiency caused by mutations of CD36 gene, providing an experimental basis for mastering the polymorphism of the mutant genes in various populations.

Based on the SNPs in CD36 gene that cause GPIV deficiency, the disclosure provides a specific-sequence primer designed for amplifying a wild-type sequence, a specific-sequence primer designed for amplifying a mutant sequence, and a universal primer. Each SNP genotyping system performs two PCR reactions in which the wild-type sequence and the mutant-type sequence of the SNPs in CD36 gene are amplified. A nucleotide sequence of human C-reaction protein (CRP) is amplified with a pair of primer and used as an internal reference for each PCR. The optimal annealing temperature is determined and other conditions such as Mg²⁺ concentration are changed. The SNPs in CD36 gene are amplified and genotyped by PCR. All of the PCRs can be carried out under the same conditions and therefore may be performed in an integrated thermal system.

### BRIEF DESCRIPTION OF THE DRAWINGS

FIG. 1 is a diagram showing the genotype of C275T mutation site in six samples according to Example 1 of the disclosure; (S1-1 is the heterozygous genotype for C275T mutation, S1-2 to S1-6 are homozygous genotypes for 275C wild type. Note: Lane W is the wild-type target gene, and lane M is the mutant target gene);

FIG. 2 is a diagram showing the genotype of G730A mutation site in six samples according to Example 2 of the disclosure; (S2-1 is the heterozygous genotype for G730A mutation, S2-2 to S2-6 are the homozygous genotypes for 730G wild type. Note: Lane W is the wild-type target gene, and lane M is the mutant target gene);

FIG. 3 is a diagram showing the genotype of Exon-10+2 T>G mutation site in eight samples according to Example 3 of the disclosure; (S3-1 to S3-3 are the heterozygous genotypes for Exon-10+2 T>G mutation, S3-4 to S3-8 are the homozygous genotypes for Exon-10+2T wild type. Note: Lane W is the wild-type target gene, and lane M is the mutant target gene);

FIG. 4 is a diagram showing the genotype of C1123T mutation site in six samples according to Example 4 of the disclosure; (S4-1 is the heterozygous genotype for C1123T mutation, S4-2 to S4-6 are the homozygous genotypes for 1123C wild type. Note: Lane W is the wild-type target gene, and lane M is the mutant target gene);

FIG. 5 is a diagram showing the genotype of T1229C mutation site in seven samples according to Example 5 of the disclosure; (S5-1 to S5-3 are the heterozygous genotypes for T1229C mutation, S5-4 to S5-7 are the homozygous genotypes for 1229T wild type. Note: Lane W is the wild-type target gene, and lane M is the mutant target gene);

FIG. 6 is a diagram showing the genotype of 1332 ints TGAT mutation site in seven samples according to Example 6 of the disclosure; (S6-1 to S6-2 are the heterozygous genotypes for 1332 ints TGAT mutation, S6-4 to S6-7 are the homozygous genotypes for 1332 wild-type. Note: Lane W is the wild-type target gene, and lane M is the mutant target gene);

FIG. 7 shows gel electrophoresis results of G730A mutation site amplified at different final Mg²⁺ concentrations (Note: Lane W is the wild-type target gene, and lane M is the mutant target gene; S2-1 is the heterozygous genotype for 730G/A mutation and the lanes W and M show positive bands when using the genotyping kit of the disclosure; S2-2 to S2-6 are homozygous genotypes for 730G/G, for which the genotyping tests using this kit should be positive for all bands in the lane W, and negative in the lane M; the PCR system with a final concentration of Mg²⁺ of 1.5 mM results in better and more accurate amplification results);

FIG. 8 shows gel electrophoresis results of C1123T mutation site amplified at different final Mg²⁺ concentrations (Note: Lane W is the wild-type target gene, and lane M is the mutant target gene; S4-1 is the heterozygous genotype for 1123C/T mutation and the lanes W and M show positive bands when using the genotyping kit of the disclosure; S4-2 to S4-6 are homozygous genotypes for 1123C/C, for which the genotyping tests using this kit should be positive for all bands in the lane W, and negative in the lane M; the PCR system with a final concentration of Mg²⁺ of 1.5 mM results in better and more accurate amplification results);

FIG. 9 shows gel electrophoresis results of T1229C mutation site amplified at different final Mg²⁺ concentrations (Note: Lane W is the wild-type target gene, and lane M is the mutant target gene; S5-1 to S5-2 is the heterozygous genotypes for 1229T/C mutation and the lanes W and M show positive bands when using the genotyping kit of the disclosure; S5-4 to S5-6 are homozygous genotypes for 1229T/T, for which the genotyping tests using this kit should be positive for all bands in the lane W, and negative in the lane M; the PCR system with a final concentration of Mg²⁺ of 3.5 mM results in better and more accurate amplification results);

FIG. 10 shows gel electrophoresis results of 1332 ints TGAT mutation site amplified at different final Mg²⁺ concentrations (Note: Lane W is the wild-type target gene, and lane M is the mutant target gene; S6-1 is the heterozygous genotype for the 1332 ints TGAT mutation and the lanes W and M show positive bands when using the genotyping kit of the disclosure; S6-4 is the homozygous genotype without TGAT insertion, for which the genotyping tests using this kit should be positive for all bands in the lane W, and negative in the lane M; the PCR system with a final concentration of Mg²⁺ of 3.5 mM results in better and more accurate amplification results).

### DETAILED DESCRIPTION

To further illustrate the disclosure, embodiments detailing a genotyping kit for detection of six CD36 mutant genes that encode GPIV deficiency are described below. It should be noted that the following embodiments are intended to describe and not to limit the disclosure.

The disclosure includes 20 PCR primers, which are synthesized by Shanghai Jierui Bio-Engineering Co., Ltd. Taq DNA Polmerase (produced by TaKaRa) is used for PCR amplification.

### Example 1

In this example, by using the genotyping kit of the disclosure, an implementation of genotyping for individuals whose GPIV deficiency are due to CD36 C275T (Thr92Met) mutation is specifically described.

Three primers were used: a sequence-specific primer GPIV-275a (reverse primer) configured to specifically amplify 275C wild-type sequence, a sequence-specific primer GPIV-275b (reverse primer) configured to specifically amplify 275T mutant sequence, and a forward universal primer GPIV-275c. PCR amplifications of the C275T mutation site were performed on six samples including one C275T heterozygous genotype sample and five 275C wild-type genotype samples with the three primers, which the genotype of C275T mutation site of the samples had been confirmed by DNA sequencing. A forward primer CRP I and a reverse primer CRP II were added to each PCR, thereby amplifying the nucleotide sequence of human C-reactive protein (CRP) used as an internal reference. Amplification was completed on a thermocycler (ABI PCR system 9700). In Example 1, system A was selected and its components are as follows:

| | | |
|---|---|---|
| H₂O | 4.895 | μL |
| 10 × Buffer (Mg²⁺ free) | 1 | μL |
| MgCl₂ (25 mM) | 0.8 | μL |
| dNTPs (2.5 mM each) | 0.8 | μL |
| rTaq DNA Polmerase (Takara) | 0.075 | μL |
| CRPI + CRPII (5 μM each) | 0.15 | μL |
| Sequence-specific primer GPIV-275a | 0.5 | μL |
| or GPIV-275b (10 μM) | | |
| Universal primer GPIV-275c (10 μM) | 0.5 | μL |
| DNA | 1 | μL |
| Cresol Red (10 mg/mL) | 0.04 | μL |
| Glycerine 50% (v/v) | 0.24 | μL |

(Note: The sequence-specific primer GPIV-275a was added to the 275C wild-type PCR system; the sequence-specific primer GPIV-275b was added to the 275T mutant PCR system.)

PCR amplification is carried out under the PCR cycling and running parameters: 95° C. for 5 min, 25 cycles of 95° C. for 30 sec, 68° C.-0.4° C./cycle for 30 sec, and 72° C. for 30 sec, 15 cycles of 95° C. for 30 sec, 54° C. for 30 sec, and 72° C. for 30 sec, with the final elongation step at 72° C. for 5 min, followed by an infinite hold at 12° C.

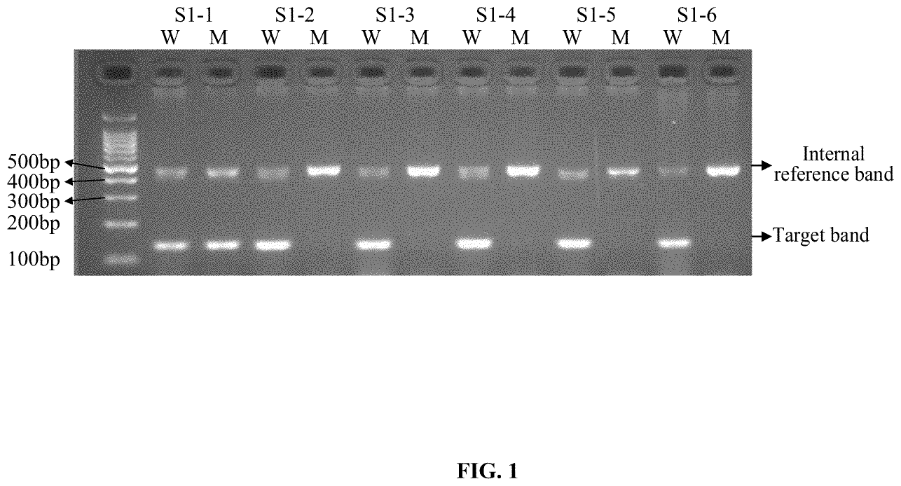

8 μL of PCR products were electrophoresed on a 2% agarose gel (containing 5% DNAGREEN provided by Beijing Tian Enze Gene Technology Co., Ltd.) The specific PCR product was observed in a gel imaging system. The gel electrophoresis results showed a clear specific band for the specific PCR product. Referring to FIG. 1, the sample S1-1 was heterozygous for the C275T mutant and therefore the target bands of wild-type and the mutant type were amplified in the wild-type lane (W) and the mutant lane (M), respectively. The samples S1-2 to S1-6 are homozygous for the 275C wild type and therefore just target band was amplified in the wild-type lane (W) and no target band was amplified in the mutant lane (M).

### Example 2

In this example, by using the genotyping kit of the disclosure, an implementation of genotyping for individuals whose GPIV deficiency are due to CD36 G730A (Asp244Asn) mutation is specifically described.

Three primers were used: a sequence-specific primer GPIV-730a (forward primer) configured to specifically amplify a 730G wild-type sequence, a sequence-specific primer GPIV-730b (forward primer) configured to specifically amplify a 730A mutant sequence, and a reverse universal primer GPIV-730c. PCR amplifications of the G730A mutation site were performed on six samples including one G730A heterozygous genotype sample and five 730G wild-type genotype samples with the three primers, which the genotype of G730A mutation site of the samples had been confirmed by DNA sequencing. A forward primer CRP I and a reverse primer CRP II were added to each PCR, thereby amplifying the nucleotide sequence of human C-reactive protein (CRP) as an internal reference. Amplification was completed on a thermocycler (ABI PCR system 9700). In Example 2, system C was selected and its components are as follows:

| | | |
|---|---|---|
| H₂O | 5.095 | μL |
| 10 × Buffer (Mg²⁺ free) | 1 | μL |
| MgCl₂ (25 mM) | 0.6 | μL |
| dNTPs (2.5 mM each) | 0.8 | μL |
| rTaq DNA Polmerase (Takara) | 0.075 | μL |
| CRPI + CRPII (5 μM each) | 0.15 | μL |
| Sequence-specific primer GPIV-730a | 0.5 | μL |
| or GPIV-730b (10 μM) | | |
| Universal primer GPIV-275c (10 μM) | 0.5 | μL |
| DNA | 1 | μL |
| Cresol Red (10 mg/mL) | 0.04 | μL |
| Glycerine 50% (v/v) | 0.24 | μL |

(Note: The sequence-specific primer GPIV-730a was added to the 730G wild-type PCR system; the sequence-specific primer GPIV-730b was added to the 730A mutant PCR system.)

The PCR cycling and running parameters were the same as those in Example 1.

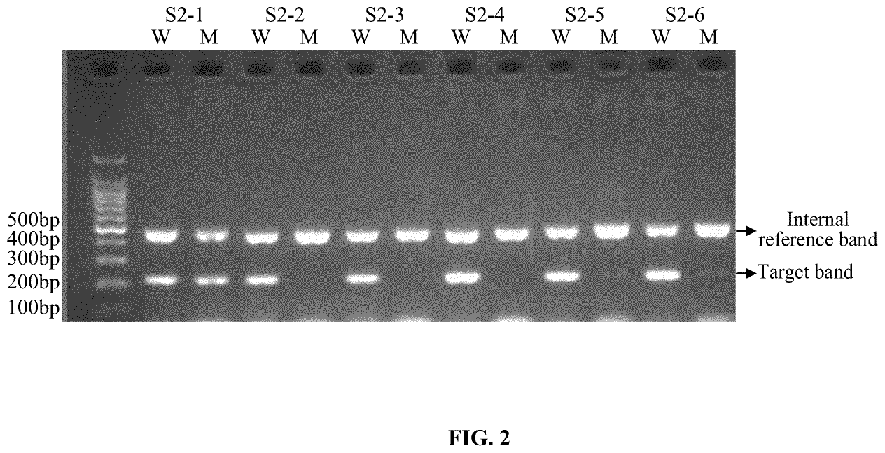

8 μL of PCR products were electrophoresed on a 2% agarose gel (containing 5% DNAGREEN provided by Beijing Tian Enze Gene Technology Co., Ltd.) The specific PCR product was observed in a gel imaging system. The gel electrophoresis results showed a clear specific band for the specific PCR product. Referring to FIG. 2, the sample S2-1 was heterozygous for the G730A mutant and therefore the target bands of wild-type and the mutant type were amplified in the wild-type lane (W) and the mutant lane (M), respectively. The samples S2-2 to S2-6 are homozygous for the 730G wild type and therefore just target band was amplified in the wild-type lane (W) and no target band was amplified in the mutant lane (M).

### Example 3

In this example, by using the genotyping kit of the disclosure, an implementation of genotyping for individuals whose GPIV deficiency are due to Exon-10+2T>G (Change in splicing site) mutation is specifically described.

Three primers were used: a sequence-specific primer GPIV-E10(+2)a (forward primer) configured to specifically amplify an Exon-10+2T wild-type sequence, a sequence-specific primer GPIV-E10(+2)b (forward primer) configured to specifically amplify an Exon-10+2G mutant sequence, and a reverse universal primer GPIV-E10(+2)c. PCR amplifications of the Exon-10+2T>G mutation site were performed on eight samples including three Exon-10+2T>G heterozygous genotype samples and five Exon-10+2T wild-type genotype samples with the three primers, which the genotype of Exon-10+2T>G mutation site of the samples had been confirmed by DNA sequencing. A forward primer CRP I and a reverse primer CRP II were added to each PCR, thereby amplifying the nucleotide sequence of human C-reactive protein (CRP) as an internal reference. Amplification was completed on a thermocycler (ABI PCR system 9700). In Example 3, system A was selected and its components are as follows:

| | | |
|---|---|---|
| H₂O | 4.895 | μL |
| 10 × Buffer (Mg²⁺ free) | 1 | μL |
| MgCl₂ (25 mM) | 0.8 | μL |
| dNTPs (2.5 mM each) | 0.8 | μL |
| rTaq DNA Polmerase (Takara) | 0.075 | μL |
| CRPI + CRPII (5 μM each) | 0.15 | μL |
| Sequence-specific primer GPIV-E10(+2)a | 0.5 | μL |
| or GPIV-E10(+2)b (10 μM) | | |
| Universal primer GPIV-E10(+2)c (10 μM) | 0.5 | μL |
| DNA | 1 | μL |
| Cresol Red (10 mg/mL) | 0.04 | μL |
| Glycerine 50% (v/v) | 0.24 | μL |

(Note: The sequence-specific primer GPIV-E10(+2)a was added to the Exon-10 + 2T wild-type PCR system; the sequence-specific primer GPIV-E10(+2)b was added to the Exon-10 + 2G mutant PCR system.)

The PCR cycling and running parameters were the same as those in Example 1.

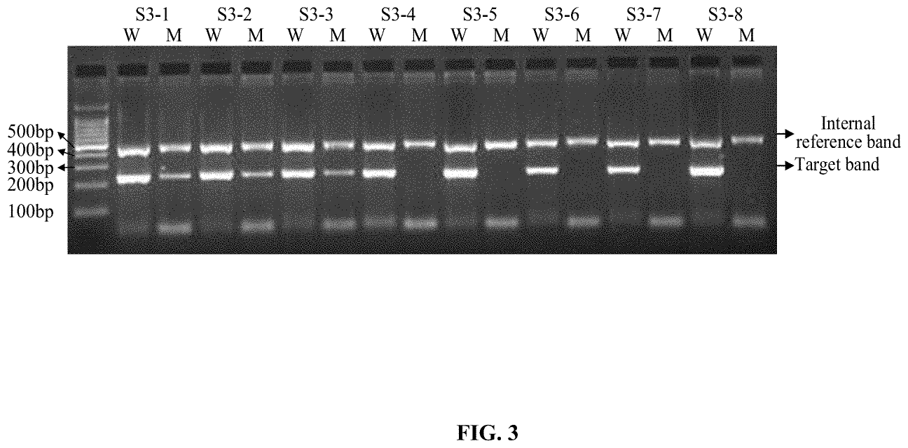

8 μL of PCR products were electrophoresed on a 2% agarose gel (containing 5% DNAGREEN provided by Beijing Tian Enze Gene Technology Co., Ltd.) The specific PCR product was observed in a gel imaging system. The gel electrophoresis results showed a clear specific band for the specific PCR product. Referring to FIG. 3, the samples S3-1 to S3-3 are heterozygous for the Exon-10+2T>G mutant and therefore the target bands of wild-type and the mutant type were amplified in the wild-type lane (W) and the mutant lane (M), respectively. The samples S3-4 to S3-8 were homozygous for the Exon-10+2T wild type and therefore just target band was amplified in the wild-type lane (W) and no target band was amplified in the mutant lane (M).

### Example 4

In this example, by using the genotyping kit of the disclosure, an implementation of genotyping for individuals whose GPIV deficiency are due to CD36 C1123T (Pro375Ser) mutation is specifically described.

Three primers were used: a sequence-specific primer GPIV-1123a (reverse primer) configured to specifically amplify a 1123C wild-type sequence, a sequence-specific primer GPIV-1123b (reverse primer) configured to specifically amplify a 1123T mutant sequence, and a forward universal primer GPIV-1123c. PCR amplifications of the C1123T mutation site were performed on six samples including one C1123T heterozygous genotype sample and five 1123C wild-type genotype samples with the three primers, which the genotype of C1123T mutation site of the samples had been confirmed by DNA sequencing. A forward primer CRP I and a reverse primer CRP II were added to each PCR, thereby amplifying the nucleotide sequence of human C-reactive protein (CRP) as an internal reference. Amplification was completed on a thermocycler (ABI PCR system 9700). In Example 4, system C was selected and its components are as follows:

| | | |
|---|---|---|
| H₂O | 5.095 | μL |
| 10 × Buffer (Mg²⁺ free) | 1 | μL |
| MgCl₂ (25 mM) | 0.6 | μL |
| dNTPs (2.5 mM each) | 0.8 | μL |
| rTaq DNA Polmerase (Takara) | 0.075 | μL |
| CRPI + CRPII (5 μM each) | 0.15 | μL |
| Sequence-specific primer GPIV-1223a | 0.5 μL | |
| or GPIV-1223b (10 μM) | | |
| Universal primer GPIV-1223c (10 μM) | 0.5 | μL |
| DNA | 1 | μL |
| Cresol Red (10 mg/mL) | 0.04 | μL |
| Glycerine 50% (v/v) | 0.24 | μL |

(Note: The sequence-specific primer GPIV-1123a was added to the 1123C wild-type PCR system; the sequence-specific primer GPIV-1123b was added to the 1123T mutant PCR system.)

The PCR cycling and running parameters were the same as those in Example 1.

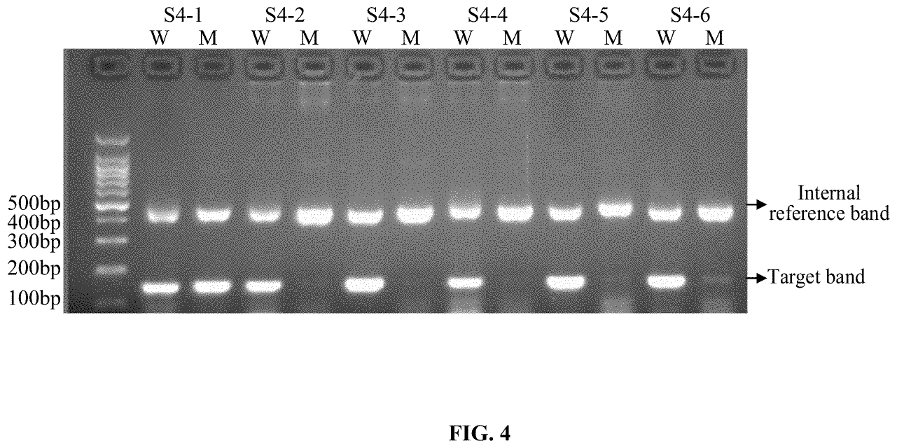

8 μL of PCR products were electrophoresed on a 2% agarose gel (containing 5% DNAGREEN provided by Beijing Tian Enze Gene Technology Co., Ltd.) The specific PCR product was observed in a gel imaging system. The gel electrophoresis results showed a clear specific band for the specific PCR product. Referring to FIG. 4, the sample S4-1 was heterozygous for the C1123T mutant and therefore the target bands of wild-type and the mutant type were amplified in the wild-type lane (W) and the mutant lane (M), respectively. The samples S4-2 to S4-6 were homozygous for the 1123T wild type and therefore just target band was amplified in the wild-type lane (W) and no target band was amplified in the mutant lane (M).

### Example 5

In this example, by using the genotyping kit of the disclosure, an implementation of genotyping for individuals whose GPIV deficiency are due to CD36 T1229C (Ile410Thr) mutation is specifically described.

Three primers were used: a sequence-specific primer GPIV-1229a (reverse primer) configured to specifically amplify a 1229T wild-type sequence, a sequence-specific primer GPIV-1229b (reverse primer) configured to specifically amplify a 1229C mutant sequence, and a forward universal primer GPIV-1229c. PCR amplifications of the T1229C mutation site were performed on seven samples including three T1229C heterozygous genotype samples and four 1229T wild-type genotype samples with the three primers, which the genotype of T1229C mutation site of the samples had been confirmed by DNA sequencing. A forward primer CRP I and a reverse primer CRP II were added to each PCR, thereby amplifying the nucleotide sequence of human C-reactive protein (CRP) as an internal reference. Amplification was completed on a thermocycler (ABI PCR system 9700). In Example 5, system B was selected and its components are as follows:

| | | |
|---|---|---|
| H₂O | 4.295 | μL |
| 10 × Buffer (Mg²⁺ free) | 1 | μL |
| MgCl₂ (25 mM) | 1.4 | μL |
| dNTPs (2.5 mM each) | 0.8 | μL |
| rTaq DNA Polmerase (Takara) | 0.075 | μL |
| CRPI + CRPII (5 μM each) | 0.15 | μL |
| Sequence-specific primer GPIV-1229a | 0.5 μL | |
| or GPIV-1229b (10 μM) | | |
| Universal primer GPIV-1229c (10 μM) | 0.5 | μL |
| DNA | 1 | μL |
| Cresol Red (10 mg/mL) | 0.04 | μL |
| Glycerine 50% (v/v) | 0.24 | μL |

(Note: The sequence-specific primer GPIV-1229a was added to the 1229T wild-type PCR system; the sequence-specific primer GPIV-1229b was added to the 1229C mutant PCR system.)

The PCR cycling and running parameters were the same as those in Example 1.

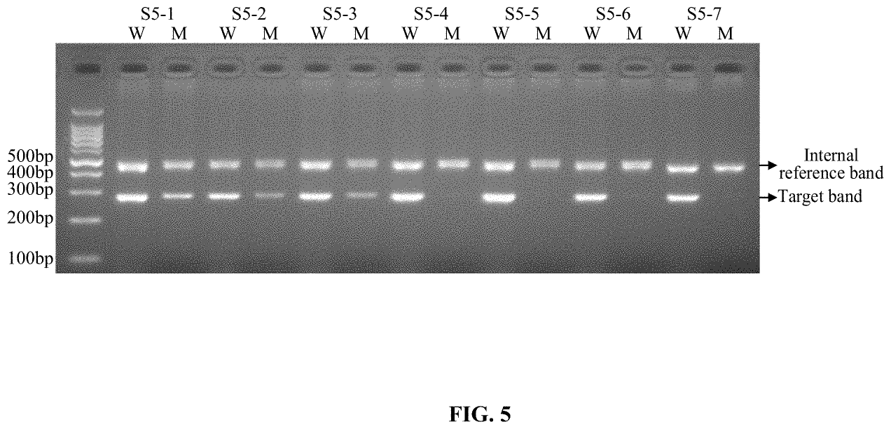

8 μL of PCR products were electrophoresed on a 2% agarose gel (containing 5% DNAGREEN provided by Beijing Tian Enze Gene Technology Co., Ltd.) The specific PCR product was observed in a gel imaging system. The gel electrophoresis results showed a clear specific band for the specific PCR product. Referring to FIG. 5, the samples S5-1 to S5-3 were heterozygous for the T1229C mutant and therefore the target bands of wild-type and the mutant type were amplified in the wild-type lane (W) and the mutant lane (M), respectively. The samples S5-4 to S5-7 were homozygous for the 1229T wild type and therefore just target band was amplified in the wild-type lane (W) and no target band was amplified in the mutant lane (M).

### Example 6

In this example, by using the genotyping kit of the disclosure, an implementation of genotyping for individuals whose GPIV deficiency are due to CD36 1332 ints TGAT mutation (frameshift at AA 445) is specifically described.

Three primers were used: a sequence-specific primer GPIV-1332a (forward primer) configured to specifically amplify a 1332 wild-type sequence, a sequence-specific primer GPIV-1332b (forward primer) configured to specifically amplify a 1332 ints TGAT mutant sequence, and a reverse universal primer GPIV-1332c. PCR amplifications of the 1332 ints TGAT mutation site were performed on seven samples including two 1332 ints TGAT heterozygous genotype samples and five 1332 wild-type genotype samples with the three primers, which the genotype of 1332 ints TGAT mutation site of the samples had been confirmed by DNA sequencing. A forward primer CRP I and a reverse primer CRP II were added to each PCR, thereby amplifying the nucleotide sequence of human C-reactive protein (CRP) as an internal reference. Amplification was completed on a thermocycler (ABI PCR system 9700). In Example 6, system B was selected and its components are as follows:

| | | |
|---|---|---|
| H₂O | 4.295 | μL |
| 10 × Buffer (Mg²⁺ free) | 1 | μL |
| MgCl₂ (25 mM) | 1.4 | μL |
| dNTPs (2.5 mM each) | 0.8 | μL |
| rTaq DNA Polmerase (Takara) | 0.075 | μL |
| CRPI + CRPII (5 μM each) | 0.15 | μL |
| Sequence-specific primer GPIV-1332a | 0.5 | μL |
| or GPIV-1332b (10 μM) | | |
| Universal primer GPIV-1332c (10 μM) | 0.5 | μL |
| DNA | 1 | μL |
| Cresol Red (10 mg/mL) | 0.04 | μL |
| Glycerine 50% (v/v) | 0.24 | μL |

(Note: The sequence-specific primer GPIV-1332a was added to the 1332 wild-type PCR system; the sequence-specific primer GPIV-1332b was added to the 1332 ints TGAT mutant PCR system.)

The PCR cycling and running parameters were the same as those in Example 1.

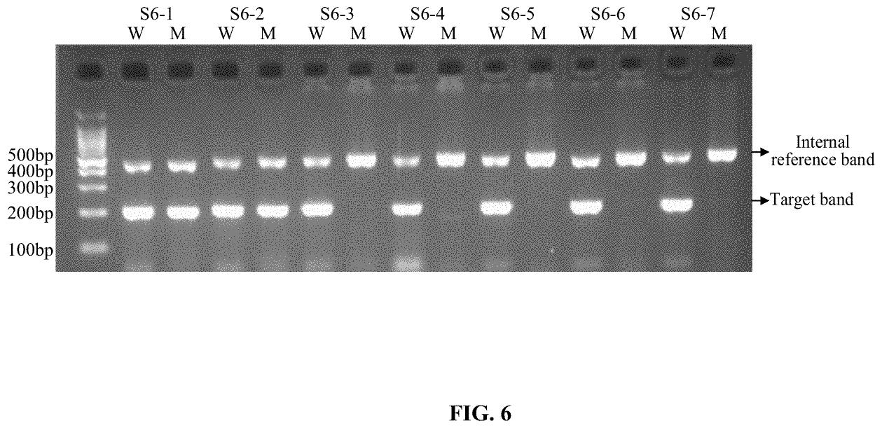

8 μL of PCR products were electrophoresed on a 2% agarose gel (containing 5% DNAGREEN provided by Beijing Tian Enze Gene Technology Co., Ltd.) The specific PCR product was observed in a gel imaging system. The gel electrophoresis results showed a clear specific band for the specific PCR product. Referring to FIG. 6, the samples S6-1 to S6-2 were heterozygous for the 1332 ints TGAT mutant and therefore the target bands of wild-type and the mutant type were amplified in the wild-type lane (W) and the mutant lane (M), respectively. The samples S6-3 to S6-7 were homozygous for the 1332 wild type and therefore just target band was amplified in the wild-type lane (W) and no target band was amplified in the mutant lane (M).

### Example 7

The influence of the final Mg²⁺ concentration in the PCR system on the test results of the kit.

1. The influence of different final Mg²⁺ concentration on amplification of the sequence with regard to G730A mutation.

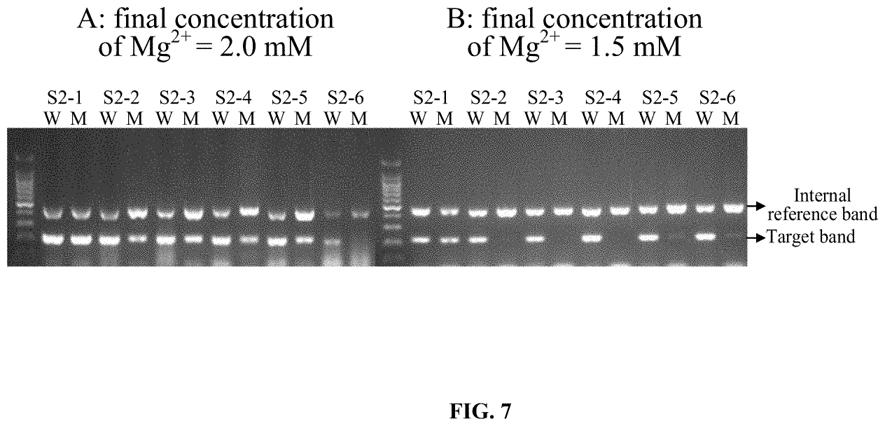

In some samples (S2-2 to S2-5) are homozygous for wild type 730G/G. When the final concentration of Mg²⁺ was 2.0 mM, a non-specific product was amplified at the location of the target band in the 730A mutant PCR system, causing false positive results and affecting the judgment of the results. When the final Mg²⁺ concentration was 1.5 mM, the non-specific product was very weak or absent in the 730A mutant PCR system, without affecting the amplification of wild-type target band. Referring to FIG. 7, the samples with G730A mutation can also be detected by PCR, thereby giving a correct interpretation of the result.

2. The influence of different final Mg²⁺ concentration on amplification of C1123T mutation.

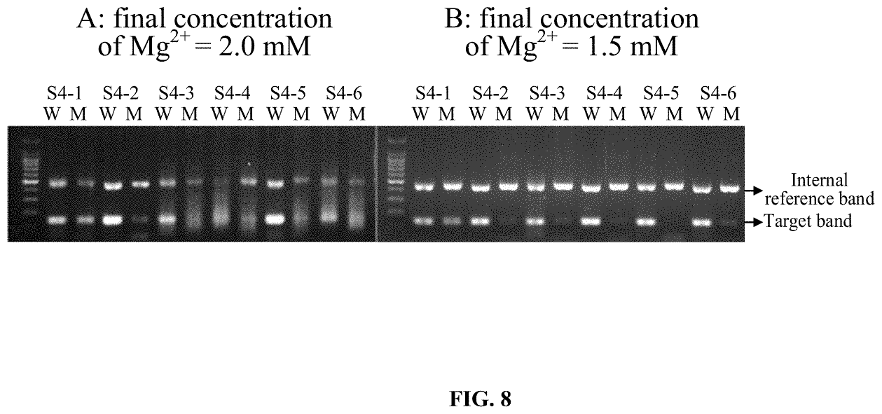

In some samples (S4-2 to S4-6) were homozygous for wild type 1123C/C. When the final concentration of Mg²⁺ was 2.0 mM, a weak non-specific product was amplified at the location of the target band in the 1123T mutant PCR system, producing poor amplification effect and affecting the judgment of the results. When the final Mg²⁺ concentration was 1.5 mM, the non-specific product was very weak or absent in the 1123T mutant PCR system, without affecting the amplification of wild-type target band. Referring to FIG. 8, the samples with 1123T mutation can also be detected by PCR, thereby giving a correct interpretation of the result.

3. The influence of different final Mg²⁺ concentration on amplification of T1229C mutation.

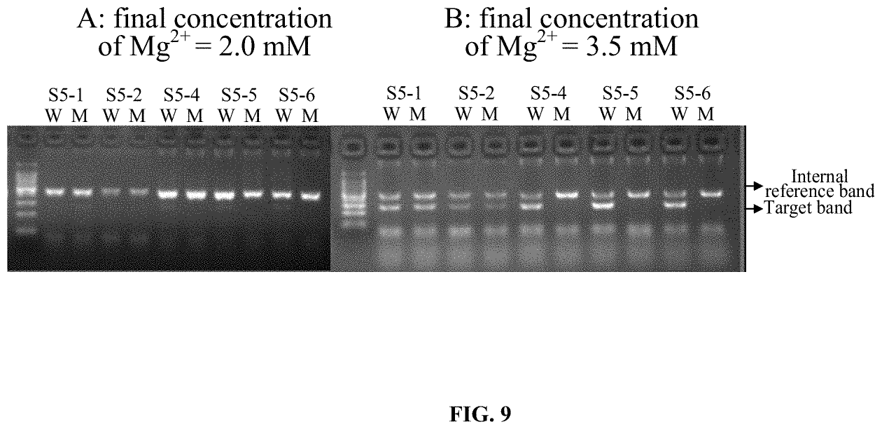

When the final concentration of Mg²⁺ was 2.0 mM, the sequences with regard to the T1229C mutant and wild type site cannot be respectively amplified from the T1229C mutant heterozygous sample DNA (S5-1, S5-2) and the wild-type sample DNA (S5-4 to S5-6). Referring to FIG. 9, when the final Mg²⁺ concentration was 3.5 mM, the target band was successfully amplified, giving a more accurate interpretation of the result.

4. The influence of different final Mg²⁺ concentration on amplification of 1332 ints TGAT mutation.

The sample S6-1 was heterozygous for 1332 ints TGAT mutant and genotyped by the kit of the disclosure. The target bands should be positive in the lanes W and M. The sample S-4 was homozygous for the 1332 wild-type that without TGAT insertion at position 1332 and was genotyped by the kit of the disclosure. The target bands should be positive in the lane W and negative in the lanes M.

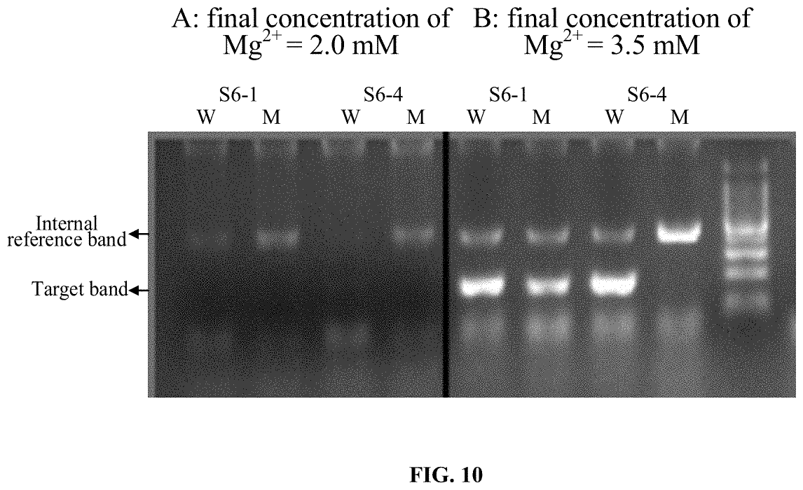

When the final concentration of Mg²⁺ was 2.0 mM, the sequences with regard to the 1332 ints TGAT mutant and wild type site cannot be respectively amplified from the DNAs released from the 1332 ints TGAT mutant heterozygous sample (S6-1) and the wild-type sample (S6-4). Referring to FIG. 10, when the final concentration of Mg²⁺ was 3.5 mM, the target band was amplified, giving a more accurate interpretation of the result.

It will be obvious to those skilled in the art that changes and modifications may be made, and therefore, the aim in the appended claims is to cover all such changes and modifications.

What is claimed is:

1. A genotyping kit for detection of six CD36 mutant genes that encode GPIV deficiency, the kit comprising: six primers configured to amplify a sequence of CD36 wild-type gene, six primers configured to amplify a sequence of CD36 mutant genes, and six universal primers; wherein the mutations of CD36 gene comprises following mutation sites: C275T (Thr92Met), 730G>A (Asp244Asn), Exon-10+2 T>G (Change in splicing site), 1123C>T (Pro375Ser), 1229T>C (Ile410Thr), and 1332 ints TGAT (frameshift at AA 445).

2. The kit of claim 1, wherein:
a first primer configured to amplify a 275C wild-type sequence with regard to C275T mutation site is represented by SEQ ID NO: 1; a second primer configured to amplify a 275T mutant sequence with regard to the C275T mutation site is represented by SEQ ID NO: 2; and a first universal primer is represented by SEQ ID NO: 3;
a third primer configured to amplify a 730G wild-type sequence with regard to G730A mutation site is represented by SEQ ID NO: 4; a fourth primer configured to amplify a 730A mutant sequence with regard to the G730A mutation site is represented by SEQ ID NO: 5; and a second universal primer is represented by SEQ ID NO: 6;
a fifth primer configured to amplify a wild-type (Exon-10+2T) sequence with regard to Exon-10+2T>G mutation site is represented by SEQ ID NO: 7; a sixth primer configured to amplify a mutant (Exon-10+2G) sequence with regard to the Exon-10+2T>G mutation site is represented by SEQ ID NO: 8; and a third universal primer is represented by SEQ ID NO: 9;
a seventh primer configured to amplify a 1123C wild-type sequence with regard to C1123T mutation site is represented by SEQ ID NO: 10; an eighth primer configured to amplify a 1123T mutant sequence with regard to the C1123T mutation site is represented by SEQ ID NO: 11; and a fourth universal primer is represented by SEQ ID NO: 12;
a ninth primer configured to amplify a wild-type (1229T) sequence with regard to T1229C mutation site is represented by SEQ ID NO: 13; a tenth primer configured to amplify a 1229C mutant sequence with regard to the T1229C mutation site is represented by SEQ ID NO: 14; and a fifth universal primer is represented by SEQ ID NO: 15; and
an eleventh primer configured to amplify a wild-type sequence without TGAT insertion at position 1332 with regard to 1332 ints TGAT mutation site is represented by SEQ ID NO: 16; a twelfth primer configured to amplify a mutant sequence with TGAT insertion at position 1332 with regard to 1332 ints TGAT mutation site is represented by SEQ ID NO: 17; and a sixth universal primer is represented by SEQ ID NO: 18.

3. The kit of claim 2, wherein the kit further comprises C-reactive protein (CRP) as an internal reference; a forward primer for the internal reference is represented by SEQ ID NO: 19, and a reverse primer for the internal reference is represented by SEQ ID NO: 20.

4. The kit of claim 1, wherein the kit further comprises magnesium chloride as a PCR reagent; and a final concentration of magnesium chloride in a PCR system is 1.5-3.5 mM.

5. The kit of claim 2, wherein the kit further comprises magnesium chloride as a PCR reagent; and a final concentration of magnesium chloride in a PCR system is 1.5-3.5 mM.

6. The kit of claim 4, wherein in each wild-type/mutant PCR system, the final concentration of magnesium ion for detecting mutations of C275T and Exon-10 (+2 T>G) is 2 mM; and a final volume of the PCR system is 10 μL.

7. The kit of claim 5, wherein in each wild-type/mutant PCR system, the final concentration of magnesium ion for detecting mutations of C275T and Exon-10 (+2 T>G) is 2 mM; and a final volume of the PCR system is 10 μL.

8. The kit of claim 4, wherein in each wild-type/mutant PCR system, the final concentration of magnesium ion for detecting mutations of T1229C and 1332 ints TGAT is 3.5 mM; and a final volume of the PCR system is 10 μL.

9. The kit of claim 5, wherein in each wild-type/mutant PCR system, the final concentration of magnesium ion for detecting mutations of T1229C and 1332 ints TGAT is 3.5 mM; and a final volume of the PCR system is 10 μL.

10. The kit of claim 4, wherein in each wild-type/mutant PCR system, the final concentration of magnesium ion for detecting mutations of G730A and C1123T is 1.5 mM; and a final volume of the PCR system is 10 μL.

11. The kit of claim 5, wherein in each wild-type/mutant PCR system, the final concentration of magnesium ion for detecting mutations of G730A and C1123T is 1.5 mM; and a final volume of the PCR system is 10 μL.

12. The kit of claim 1, wherein the kit further comprises dNTPs, 10×Buffer, DNA Polmerase, 10 mg/mL Cresol Red, and 50% (v/v) Glycerine.

13. The kit of claim 2, wherein the kit further comprises dNTPs, 10×Buffer, DNA Polmerase, 10 mg/mL Cresol Red, and 50% (v/v) Glycerine.

14. The kit of claim 1, wherein a PCR amplification for genotyping of mutations of CD36 gene is carried out under the following conditions:
95° C. for 5 min;
25 cycles under following conditions: 95° C. for 30 sec, 68° C.-0.4° C./cycle for 30 sec, and 72° C. for 30 sec;
15 cycles under following conditions: 95° C. for 30 sec, 54° C. for 30 sec, and 72° C. for 30 sec;
elongation at 72° C. for 5 min; and
storage at 12° C.
